(19) United States
(12) Patent Application Publication
(10) Pub. No.: US 2026/0215839 A1
(43) Pub. Date: Jul. 30, 2026

(54) MITIGATING NEEDLE TRACT SEEDING IN ABLATION PROCEDURES

(71) Applicant: Neuwave Medical, Inc., Madison, WI (US)

(72) Inventor: Matthew SCHANING, Cambridge, WI (US)

(73) Assignee: Neuwave Medical, Inc., Madison, WI (US)

(21) Appl. No.: 19/040,624

(22) Filed: Jan. 29, 2025

(51) Int. Cl. A61B 18/14; A61B 34/00; A61B 34/20; A61B 18/00; A61B 18/18

(52) U.S. Cl. CPC A61B 18/1477; A61B 34/20; A61B 34/25; A61B 2018/00577; A61B 2018/00595; A61B 2018/00702; A61B 18/1815; A61B 2018/1869

(57) ABSTRACT

A system is disclosed that comprises an ablation probe including an antenna and a controller in operable communication with the ablation probe and a display. The controller is operable to display, on the display, an image of the ablation probe in a needle tract in the patient, control the antenna to emit energy in the needle tract, and display, on the image, portions of the needle tract expected to have failed to receive energy from the antenna.

### BACKGROUND

The present disclosure is related to systems and methods for delivering energy to tissue for an ablation operation and, more particularly, to systems and methods for visualizing needle tracts that may be susceptible to tumor seeding.

An ablation procedure with an ablation probe typically includes two periods. The first period is an ablation period in which an antenna of the ablation probe is maintained in place within a needle tract in a patient and an ablation zone is generated to ablate target tissue. The second period is a cauterization period in which energy is emitted from the antenna as the ablation probe is slowly withdrawn from the patient to destroy seed tumor cells along the needle tract.

Although needle tract cauterization is a known practice to prevent tract seeding, a physician may inadvertently forget to cauterize the needle tract, especially in cases where multiple ablation probes are used and/or cases where probe placement is challenging and requires probe repositioning.

Accordingly, systems and methods for visualizing needle tracts that require cauterization are desirable.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, without departing from the scope of this disclosure.

FIG. 1 is a block diagram of an energy delivery system including an energy delivery device and a display, in accordance with at least one aspect of the present disclosure.

FIG. 2 is a schematic diagram of the energy delivery device of FIG. 1, in accordance with at least one aspect of the present disclosure.

FIG. 3 is an enlarged, cross-sectional view of a portion of the energy delivery device of FIG. 2, in accordance with at least one aspect of the present disclosure.

FIG. 4 is a first example image shown on the display of FIG. 1 and including an ablation target, according to at least one aspect of the present disclosure.

FIG. 5 is a second example image shown on the display of FIG. 1 and including an ablation probe inserted to a first position in a needle tract relative to the ablation target of FIG. 4, according to at least one aspect of the present disclosure.

FIG. 6 is a third example image shown on the display of FIG. 1 and including the ablation probe of FIG. 5 moved to a second position in the needle tract, according to at least one aspect of the present disclosure.

FIG. 7 is the third image of FIG. 6 with portions of the needle tract highlighted, according to at least one aspect of the present disclosure.

FIG. 8 is a fourth example image shown on the display of FIG. 1 and displaying the needle tract of FIG. 7 with first and second portions thereof highlighted, according to at least one aspect of the present disclosure.

FIG. 9 is a first example image shown on the display of FIG. 1 and displaying an ablation target, according to at least one aspect of the present disclosure.

FIG. 10 is a second example image shown on the display of FIG. 1 and displaying an ablation probe inserted in a first needle tract relative to the ablation target of FIG. 9, according to at least one aspect of the present disclosure.

FIG. 11 is a third example image shown on the display of FIG. 1 and displaying the ablation probe of FIG. 10 moved into a second needle tract, according to at least one aspect of the present disclosure.

FIG. 12 is the third image of FIG. 11 with portions of the first and second needle tracts highlighted, according to at least one aspect of the present disclosure.

FIG. 13 is a three-dimensional image of the display shown in FIG. 12, according to at least one aspect of the present disclosure.

FIG. 14 is an example fourth image shown on the display of FIG. 1 and displaying the needle tracts of FIG. 12 with first and second portions thereof highlighted, according to at least one aspect of the present disclosure.

### DETAILED DESCRIPTION

The present disclosure is related to systems and methods for delivering energy to tissue for an ablation operation and, more particularly, to systems and methods for visualizing needle tracts that may be susceptible to tumor seeding.

The present disclosure is related to comprehensive systems, devices, and methods for delivering energy (e.g., microwave energy, radiofrequency energy, laser, focused ultrasound, plasma, etc.) to tissue for a wide variety of applications including medical procedures (e.g., percutaneous or surgical). Example medical procedures that may benefit from the embodiments described herein include, but are not limited to, tissue ablation, resection, cautery, vascular thrombosis, intraluminal ablation of a hollow viscus, cardiac ablation for treatment of arrhythmias, electrosurgery, tissue harvest, cosmetic surgery, intraocular use, or any combination thereof.

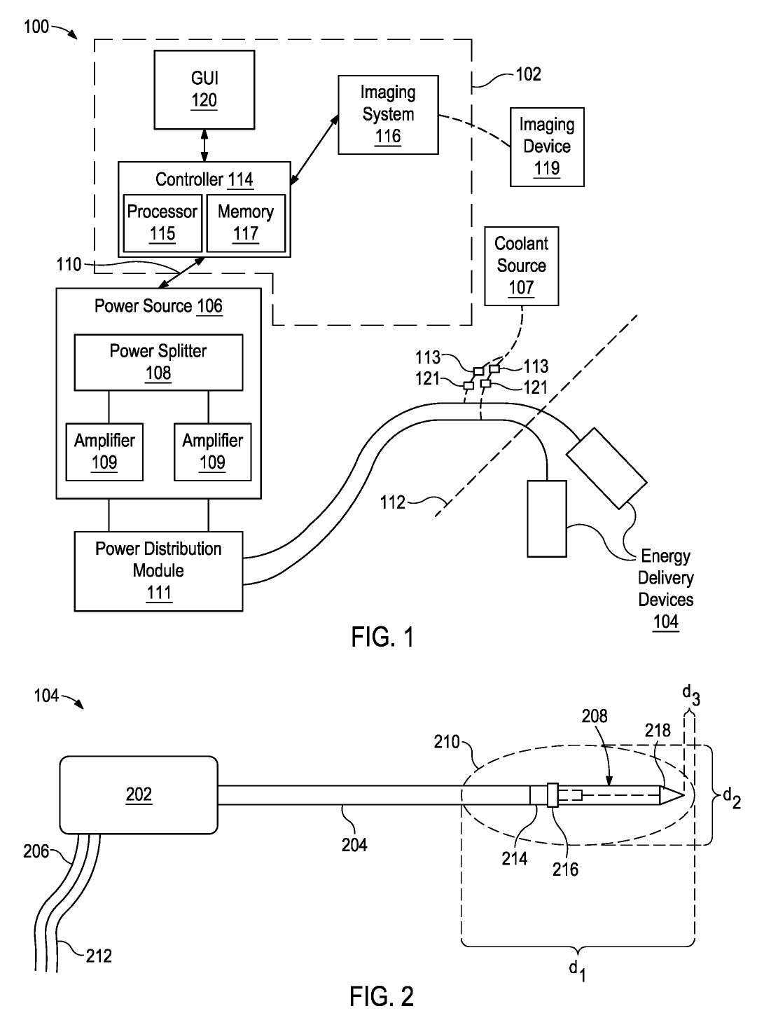

FIG. 1 is a block diagram of an energy delivery system 100, in accordance with at least one aspect of the present disclosure. As illustrated, the energy delivery system 100 (hereafter “the system 100”) may include a control system 102 and one or more energy delivery devices or “ablation probes” 104 (two shown) designed to deliver (emit) energy to a target tissue region of a patient. While two ablation probes 104 are shown, the system 100 may include only one ablation probe 104 or more than two ablation probes 104 (e.g. three, four, or five ablation probes).

The system 100 may further include a power source or generator 106 communicably coupled to the control system 102 and the ablation probes 104 to direct, control, and deliver (provide) electrical power thereto. The power source 106 may include a power splitter 108 that receives power from an external power source (e.g. a wall outlet) and directs power to one or more amplifiers 109 (two shown), which may amplify the voltage, current, or power from the power splitter 108 to an associated ablation probe 104. While two amplifiers 109 are shown, each associated with a corresponding ablation probe 104, the power source 106 may include less than two amplifiers (e.g. one amplifier) or more than two amplifiers (e.g. three, four, or five amplifiers, for example). Each amplifier 109 may be coupled to a corresponding ablation probe 104 via a power distribution module 111, which may provide strain relief to cabling extending from the amplifiers 109 to the ablation probes 104. The power distribution module 111 may be coupled to a structure in the operating room, such as a surgical bed, and may house connection hardware of the probes 104.

The power source 106 may supply energy required to operate various components of the system 100. The power source 106 may also supply energy to the ablation probes 104, such as microwave energy, radiofrequency energy, radiation, cryo energy, electroporation, high intensity focused ultrasound, or any combination thereof. In accordance with principles of the present disclosure, the power source 106 may supply microwave energy to the ablation probes 104 for purposes of tissue ablation. More specifically, power may be supplied to the ablation probes 104, but the microwave energy may be generated in a microwave generator and sent to the ablation probe 104. The power source 106 may include one or more energy generators configured to provide as much as 140-150 watts of microwave power at a frequency from 915 MHz to 5.8 GHz, although the present disclosure is not so limited. The power splitter 108 may comprise a power distribution system operable to distribute the energy from the power source 106 to the ablation probes 104. The power splitter 108 may be configured to provide varying energy levels to different regions of the ablation probes 104.

The control system 102 may monitor, control, and provide feedback concerning operation of the system 100. As illustrated, the control system 102 may include a controller 114, an imaging system 116, and a graphical user interface (GUI) or display 120, such as a touchscreen interface, which can be accessed by a user (e.g., a surgeon, a nurse, bedside assist, etc.) to operate the system 100. In some applications, the control system 102 may be mounted to or otherwise form part of a portable cart or “procedure cart,” and the GUI 120 may be arranged in a display region for operating and/or monitoring the components of the system 100.

The controller 114 may include a processor 115 and a memory or memory device 117 comprising any storage media readable by the processor 115. The memory 117 may store software or software instructions executable by the processor 115 to carry out functions and operations of the system 100. Examples of the memory 117 include, but are not limited to, random access memory (RAM), read-only memory (ROM), computer chips, optical discs (e.g., compact discs (CDs), digital video discs (DVDs), etc.), magnetic disks (e.g., hard disk drives (HDDs), floppy disks, ZIP® disks, etc.), magnetic tape, and solid state storage devices (e.g., memory cards, “flash” media, etc.). As used herein, the term “computer readable medium” refers to any device or system for storing and providing information (e.g., data and instructions) to the processor 115. Examples of computer readable media include, but are not limited to, optical discs, magnetic disks, magnetic tape, solid-state media, and servers for streaming media over networks.

Based on instructions provided by the software, the controller 114 may be configured to regulate the amount of energy (e.g., microwave energy) provided to a tissue region by the ablation probes 104 by monitoring characteristics of the tissue region, such as the size and shape of a target tissue, the temperature of the tissue region, etc. The controller 114 interacts with the ablation probes 104 to raise or lower (e.g., tune) the amount of energy delivered to the tissue region. The controller 114 may also be configured to prime coolants for distribution into the ablation probes 104 such that the coolant is delivered at a desired temperature, as discussed in more detail below.

In some applications, the type of tissue being treated is inputted into the software for purposes of allowing the controller 114 to regulate (e.g., tune) the delivery of microwave energy to the tissue region based upon pre-calibrated methods for that particular type of tissue or tissue region. In other embodiments, however, the type of probe selected for the particular procedure may be specifically tuned to a specific tissue type, and projected (expected) ablation sizes may be based on tissue type. In such embodiments, the controller 114 may not control power delivery based on tissue type. In yet other embodiments, the controller 114 generates a chart or diagram based upon a particular type of tissue or tissue region displaying characteristics useful to a user of the system.

The controller 114 may allow a user to choose power, duration of treatment, different treatment algorithms for different tissue types, simultaneous application of power to multiple probes 104, coherent and incoherent phasing, etc. The controller 114 may also be configured to create a database of information (e.g., required energy levels, duration of treatment for a tissue region based on particular patient characteristics, etc.) pertaining to ablation treatments for a particular tissue region based upon previous treatments with similar or dissimilar patient characteristics.

The imaging system 116 may be in communication with the controller 114 and comprise one or more imaging devices 119. Example imaging devices include, but are not limited to, ultrasound transducers, endoscopic devices, stereotactic computer assisted neurosurgical navigation devices, thermal sensor positioning systems, motion rate sensors, steering wire systems, intraprocedural ultrasound, interstitial ultrasound, microwave imaging, acoustic tomography, dual energy imaging, fluoroscopy, computerized tomography magnetic resonance imaging, nuclear medicine imaging devices triangulation imaging, thermoacoustic imaging, infrared and/or laser imaging, or electromagnetic imaging. In some embodiments, the system 100 uses endoscopic cameras, imaging components, and/or navigation systems that permit or assist in placement, positioning, and/or monitoring of the ablation probes 104.

The imaging system 116 may be configured to monitor ablation procedures, such as a position of the ablation probes 104 within a patient, as described in more detail below, and/or the amount of ablation occurring within a particular tissue region(s) undergoing a thermal ablation procedure. The monitoring includes, but is not limited to, MRI imaging, CT imaging, ultrasound imaging, nuclear medicine imaging, and fluoroscopy imaging. The software may be designed to automatically obtain images of a tissue region (e.g., MRI imaging, CT imaging, ultrasound imaging, nuclear medicine imaging, fluoroscopy imaging), automatically detect any changes in the tissue region (e.g., blood perfusion, temperature, amount of necrotic tissue, etc.), and based on the detection to automatically adjust the amount of energy delivered to the tissue region through the ablation probes 104.

The components of the system 100 may be connected via one or more cables or transmission lines 110. Moreover, the ablation probes 104 are designed to operate within a sterile field facilitated by the use of a sterile field barrier 112 that separates the ablation probes 104 from the remaining components of the system 100. The sterile field barrier 112 creates the sterile field, which includes any region permitting access only to sterilized items (e.g., sterilized devices, sterilized accessory agents, sterilized body parts, etc.). The sterile field barrier 112 hinders entry of non-sterile items into the sterile field, and the ablation probes 104 are configured for operation within the sterile field.

The system 100 may further include a coolant source 107, which stores therein a cooling fluid or “coolant”. Example coolants include, but are not limited to, water, glycol, air, inert gases (e.g., helium), carbon dioxide, nitrogen, sulfur hexafluoride, ionic solutions (e.g., sodium chloride with or without potassium and other ions), dextrose in water, Ringer's lactate, organic chemical solutions (e.g., ethylene glycol, diethylene glycol, or propylene glycol), oils (e.g., mineral oils, silicone oils, fluorocarbon oils), liquid metals, freons, halomethanes, liquified propane, other haloalkanes, anhydrous ammonia, sulfur dioxide, or any combination thereof.

The system 100 may further include one or more valves 113 fluidically coupled to the coolant source 107 and a respective ablation probe 104. The valves 113 may control the flow rate and/or pressure of coolant from the coolant source 107 to the respective ablation devices 104. The valves 113 may be any suitable valve (e.g., gate, globe, ball, etc.) that is transitionable (actuatable) between an open state (e.g., 100% open), a closed state (e.g.,0% open), and a plurality of partially open states between the open and closed states (e.g.,10%, 25%, 50%, 75%, or 90%). In applications where the valves 113 are electromechanically actuatable, the valves 113 may each include a motor in operable communication with the controller 114, via a wired or wireless connection, and which function to transition the valves 113 between their respective open, closed, and partially open states. Alternatively, the valves 113 may be solenoid valves that are transitionable between their respective open, closed, and partially opened states by the controller 114. The valves 113 may be transitionable between their respective open, closed, and partially open states based on a user input provided to the GUI 120 or automatically, such as based on inputs provided to the controller 114 from various sensors of the system 100.

The system 100 may further include one or more sensors 121 for sensing one or more parameters associated with the coolant provided from the coolant source 107. The sensors 121 may include pressure sensors operable to sense a pressure of the coolant provided from the coolant source 107, flow sensors operable to sense a flow rate of the coolant provided from the coolant source 107, or temperature sensors for sensing a temperature of the coolant provided from the coolant source 107, or any combination thereof. Each valve 113 may have associated therewith one or more of the sensors 121 to sense one or more parameters of the coolant provided to the respective ablation probe 104. The controller 114 may be in operable communication with the one or more sensors 121, via a wired or wireless connection, and may receive the sensed parameters therefrom. The controller 114 may control one or more aspects of the system 100 based on the sensed parameters, such as the state of the valves 113.

FIG. 2 is a schematic diagram of an example ablation probe 104, in accordance with at least one aspect of the present disclosure. As indicated above, the ablation probe 104 may be configured to deliver (emit) energy (e.g., microwave energy, radiofrequency energy, radiation energy) to a target tissue region. As illustrated, the ablation probe 104 includes a handle or housing 202 and an elongate shaft or probe cannula 204 extending distally from the handle 202.

A cable or cable assembly 206 may be operatively coupled to the handle 202 and configured to convey electrical power thereto. The cable assembly 206 may extend from the power distribution module 111 (FIG. 1), for example, and may provide the power sufficient to operate the ablation probe 104. An antenna 208 is provided at the distal end of the probe cannula 204 and receives electrical power from the cable assembly 206 to emit energy (e.g., microwave energy) to a target tissue region and thereby generate an ablation zone 210 (shown in dashed lines).

The ablation zone 210 may include a longitudinal length d₁, a lateral width d₂(e.g. a diameter of the ablation zone 210), and a distance d₃ between a distal end of the stylet 218 and a distal-most end of the ablation zone 210. The size of the dimensions d₁, d₂, d₃ of the ablation zone 210 may depend on one or more operating parameters, such as the type of ablation probe, the power level P of the power source 106, and the amount of time T the ablation probe 104 is energized. For instance, a first type of ablation probe 104 that is energized at a first power level P₁ for a first amount of time T₁ may be expected to generate an ablation zone 210 that includes a first longitudinal length (d₁), a first lateral width (d₂) and a first distance (d₃) between the distal end of the stylet 218 and the distal-most end of the ablation zone, while a second ablation probe 104 different than the first ablation probe that is energized at a second power level P₂ different than the first power level P₁ for a second amount of time T₂ different than the first amount of time T₁ may be expected to generate an ablation zone that includes a second longitudinal length (d₁’) different from the first longitudinal length (d₁), a second lateral width (d₂’) different from the first lateral width (d₂), and a second distance (d₃’) between the distal end of the stylet 218 and the distal-most end of the ablation zone different from the first distance (d₃). It should be noted that only one of the parameters described above (e.g. type of instrument, power level, and time) may need to be adjusted to change the resulting dimensions d₁, d₂, d₃.

The memory 117 may store therein a look-up table that includes various combinations of types of ablation probes, activation times T, and power levels P that are expected to yield various sized ablation zones 210 with varying dimensions (e.g. d₁, d₂, d₃). The expected, or predicted, dimensions of the ablation zone may be based on ex-vivo data, in-vivo data, or clinical data, or combinations thereof. Accordingly, should a user desire to generate an ablation zone 210 with particular dimensions, the controller 114 may retrieve from the memory 117 a particular combination of type of ablation probe, activation time, and power level that is expected to yield an ablation zone that meets, or at least substantially comes close to, the dimensions desired of the user. Additional information regarding predicted ablation zones is discussed in U.S. Patent Application No. 18/811,266, entitled “DYNAMIC VISUALIZATION OF EXPECTED ABLATION ZONE”, filed August 21, 2024, which is hereby incorporated by reference in its entirety herein.

A cooling tube 212 may be operatively coupled to the coolant source 107 (FIG. 1), and the handle 202 and may be configured to convey the coolant from the coolant source 107 to the ablation probe 104.

The ablation probe 104 may include a sharp stylet tip or “stylet” 218 located at the distal end of the antenna 208 and otherwise forming the distal end of the ablation probe 104. The stylet 218 may facilitate percutaneous insertion of the ablation probe 104. The stylet 218 may be made of a variety of rigid or hardened materials including, but not limited to, a hardened resin, a metal (e.g., titanium or an equivalent of titanium, stainless steel, etc.), a ceramic, or any combination thereof. In at least one application, the stylet 218 may be brazed to zirconia or an equivalent of zirconia. In such applications, the stylet 218 may comprise an extension of a metal portion of the antenna 208 and may be electrically active.

The ablation probe 104 may further include a stick region 214, alternately referred to as a “tissue-loc” region, provided on the probe cannula 204 and a plug region 216 provided on the probe cannula 204 distal to the stick region 214 at or near the antenna 208. The stick and plug regions 214, 216 may be defined as portions of the probe cannula 204, and will be discussed in more detail below.

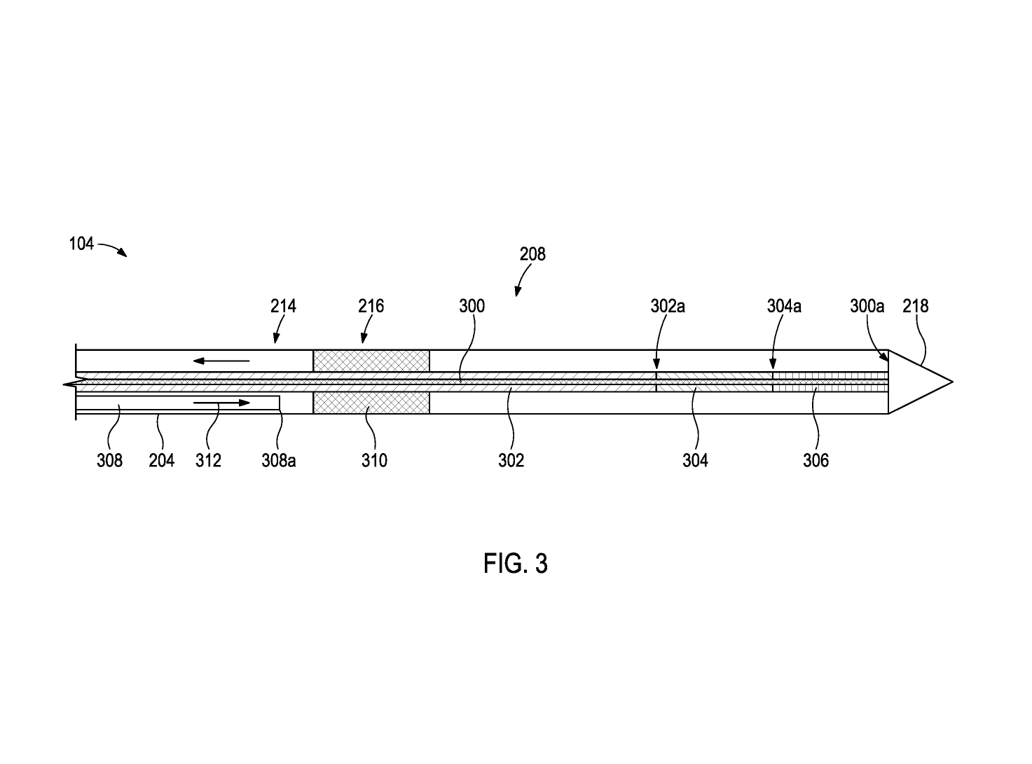

FIG. 3 is an enlarged, cross-sectional view of the probe cannula 204 and antenna 208 of the ablation probe 104, in accordance with at least one aspect of the present disclosure. The ablation probe 104 may include an inner conductor 300 and an outer conductor 302 extending through the probe cannula 204 and the antenna 208, with the outer conductor 302 being positioned about (around) the inner conductor 300 (i.e., the inner conductor 300 extends within the outer conductor 302). As illustrated, a distal end 300a of the inner conductor 300 extends distal to (beyond) a distal end 302a of the outer conductor 302. The inner and outer conductors 300, 302 may be made of a conductive material that allows current to be transmitted along their lengths thereof. The inner and outer conductors 300, 302 may be made of a metal, for example, such as stainless steel, silver, copper, brass or aluminum, or alloys thereof.

The ablation probe 104 may further include an insulator 304 extending from the distal end 302a of the outer conductor 302 and positioned about (around) the inner conductor 300. The insulator 304 may be made of a variety of non-conductive materials including, but not limited to, a ceramic or a polymer, such as polyamide, linear polyethylene (PE), polyethylene terephthalate (PET), polytetrafluoroethylene (PTFE) isotactic polypropylene (PP), or a polymer in the polyaryletherketone (PAEK) family, such as polyetheretherketone (PEEK), as examples.

The ablation probe 104 may further include a conductor load 306 extending from a distal end 304a of the insulator 304 and positioned about (around) the inner conductor 300. Accordingly, the insulator 304 axially interposes the outer conductor 302 and the conductor load 306. The conductor load 306 may be made of a metal, such as stainless steel, silver, copper, brass or aluminum, alloys thereof, or any combination thereof. The conductor load 306 may serve as a load point and may include a longitudinal length that is tuned to match the dielectric properties of the surrounding tissue.

The ablation probe 104 may further include a cooling tube 308 extending through the probe cannula 204 and terminating at the stick region 214. The cooling tube 308 may be fluidically coupled to the cooling tube 212 (FIG. 2) such that the cooling tube 308 may convey coolant 312 from the coolant source 107 (FIG. 1) and the cooling tube 212 (FIG. 2) to the stick region 214. A user may provide an input to the handle 202 (FIG. 2) and/or the GUI 120 (FIG. 1) to control flow of the coolant 312 through the cooling tube 308 to regulate a temperature of the antenna 208, the ablation zone 210, and/or the stick region 214, as discussed in more detail below. The cooling tube 308 may be made of a polymer, such as polyamide, linear polyethylene (PE), polyethylene terephthalate (PET), polytetrafluoroethylene (PTFE) isotactic polypropylene (PP), or a polymer in the polyaryletherketone (PAEK) family, such as polyetheretherketone (PEEK), as examples.

The stick region 214 is designed to attain and maintain a temperature that promotes adherence of a tissue region onto the surface of the stick region 214. More specifically, the stick region 214 may operate as an anchoring element that freezes the interface between the stick region 214 and the adjacent tissue, thereby sticking (maintaining, locking, etc.) the antenna 208 in place. In operation, coolant 312 from the coolant source 107 (FIG. 1) may be conveyed to the stick region 214 by way of the cooling tube 212 (FIG. 2) and expelled from a distal end 308a of the cooling tube 308. Upon expulsion from the distal end 308a of the cooling tube 308, the pressure of the coolant 312 quickly decreases, thereby causing the coolant 312 to correspondingly decrease in temperature (the Joule-Thompson effect). The decreased temperature of the coolant 312 causes the temperature of the stick region 214 to decrease, and the coolant 312 is recirculated back along the probe cannula 204, such as to a coolant sink, for example.

Once a threshold “low” temperature is reached at the stick region 214, contact with adjacent tissue causes the tissue to adhere (stick or couple) to the stick region 214, thereby resulting in attachment of the energy delivery device 104 to the tissue. During ablation, as the tissue warms, the antenna 208 remains secured to the tissue region due to tissue desiccation and charring. The stick region 214 may be made of any material able to attain and maintain a temperature such that contact with tissue results in adherence of the tissue onto the stick region 214. Example materials for the stick region 214 include, but are not limited to, a metal.

With reference now to FIGS. 1 and 3, the controller 114 may control a state of one or more of the valves 113, thereby controlling an amount of coolant 312 provided to the stick region 214 of the ablation probe 104. Tissue may not adhere to the stick region 214 unless a threshold flow rate and/or pressure of coolant 312 is conveyed through the cooling tube 308 to the stick region 214. For instance, the valves 113 may be transitionable between a first state and a second state. The first state may be a first, partially opened state of the valve 113, where the valve 113 is opened a first amount, and the second state may be a second, partially opened state or the open state of the valve 113, where the valve 113 is opened a second amount greater than the first amount. The first and second states of the valves 113 may be stored in the memory 117.

In the first state, a first flow rate and/or pressure of coolant 312 may be conveyed through the cooling tube 308 to the stick region 214. The first flow rate and/or pressure of coolant 312 may be insufficient to cause tissue to adhere to the stick region 214, thereby allowing the stick region 214 to move relative to the tissue positioned thereagainst. In the second state, a second flow rate and/or pressure of coolant 312 greater than the first flow rate and/or pressure of coolant 312 may be conveyed through the cooling tube 308 to the stick region 214. The second flow rate and/or pressure of coolant 312 may be sufficient to cause tissue to adhere to the stick region 214, thereby preventing the stick region 214 from moving relative to the tissue positioned thereagainst.

The ablation probe 104 may further include a seal 310, which may define the plug region 216 on the probe cannula 204. As illustrated, the seal 310 may be provided distal to the distal end 308a of the cooling tube 308 and the stick region 214 and otherwise interposing the stick region 214 and the antenna 208. The seal 310 may be configured to prevent a reduction in temperature resulting from the cooled probe cannula 204 and the stick region 214 from affecting (e.g., reducing) the temperature within the antenna 208. Accordingly, the seal 310 separates interior portions of the ablation probe 104 to prevent cooling or heating of a portion or portions of the probe 104 while permitting cooling or heating of other portions. The seal 310 may be made of an insulative material capable of being in contact with a material or region having a low temperature without having its temperature significantly reduced. Example insulative materials for the seal 310 include, but are not limited to, a synthetic polymer (e.g., polystyrene, polyicynene, polyurethane, polyisocyanurate), aerogel, fiberglass, cork, or any combination thereof.

Additional information regarding the ablation probe 104, such as the construction and function thereof, is described in U.S. Patent No. 11,638,607, entitled “ENERGY DELIVERY SYSTEMS AND USES THEREOF”, which issued on May 2, 2023, the contents of which are hereby incorporated by reference in their entirety herein.

With continued reference to FIGS. 1 and 3, the controller 114 may be operable to use the coolant source 107 and the coolant stored therein to reduce undesired heating within and along the ablation probes 104. In particular, the controller 114 may control conveyance (flow) of the coolant into and out of the cooling tube 308 via the valves 113. The controller 114 may also be configured to control conveyance of coolant to the stick region 214, as described herein above to thereby attain and maintain a temperature that accommodates adherence of tissue onto the surface of the stick region 214. In some embodiments, a user may provide an input to the controller 114, such as via the GUI 120. Based on the input, the controller 114 may control a state of one or more of the valves 113, as discussed herein above, thereby allowing the coolant source 107 to provide coolant to the stick region 214 via the cooling tubes 212, 308.

The controller 114 may also be operable to continuously or intermittently monitor the real-time temperature of the ablation probes 104. In such embodiments, the controller 114 may communicate with one or more temperature sensors (e.g., thermocouples) terminating at various points along the probe cannula 204 and/or the antenna 208 (FIG. 2) of the ablation probe 104. Consequently, localized temperature may be monitored at several points along the antenna 208 to estimate ablation status, cooling status, or safety checks. In some applications, monitoring the temperature at several points along the antenna 208 may help determine the geographical characteristics of the ablation zone 210, such as diameter, depth, length, density, width, etc., based upon the tissue type, and the amount of power used in the ablation probe 104. In other embodiments, or in addition thereto, the temperature may be measured not only at specific points along the probe cannula 204, but continuously along its entire length.

In some embodiments, the probe cannula 204 includes a plurality of temperature sensors. A first temperature sensor may be placed at, or slightly proximal to, the antenna 208 to provide real-temperature measurements of the tissue being heated by the antenna 208. A second temperature sensor may be placed at, or adjacent to, the stick region 214 to provide real-time temperature measurements of the tissue that is being cooled, and thus adhered to, the stick region 214. A third temperature sensor may be located proximal to the first and second temperature sensors along the cannula 204, such as at the point of entry into the skin, to provide real-time measurements of the patent’s skin. The control system 102 can receive the temperature measurements from the first, second, and third sensors to control the coolant systems and cooling fluids from the controller 114 to the stick region 214 and/or other cooling systems of the energy delivery device 104.

The controller 114 may also be operable to monitor the temperature of a tissue region (e.g., tissue being treated, surrounding tissue). This may prove advantageous in helping to determine the status of the procedure (e.g., the end of the procedure). The controller 114 may communicate with the plurality of temperature sensors to provide real-time temperature information to a user and display such measurements on the GUI 120. In at least one embodiment, based on the temperature data obtained by the controller 114, the controller 114 may be configured to autonomously adjust operation of the system 100 appropriately.

### Systems and Methods for Warning of Potential Needle Tract Seeding in Ablation Procedures

An ablation procedure that uses an ablation probe, like ablation probe 104, typically includes two periods. The first period is an ablation period in which the antenna 208 of the ablation probe 104 is maintained in place within a needle tract in a patient and an ablation zone 210 is generated to ablate target tissue. The needle tract is defined as the probe 104 advances to and reaches the target tissue. The second period is a cauterization period in which energy is emitted from the antenna 208 as the ablation probe 104 is slowly withdrawn from the target tissue and the patient. The emitted energy not only cauterizes the tissue along the needle tract, but also helps to destroy seed tumor cells along the needle tract.

Although needle tract cauterization is a known practice to prevent tract seeding, a physician may inadvertently forget to cauterize the needle tract, especially in cases where multiple ablation probes are used and/or cases where probe placement is challenging and requires probe repositioning. Accordingly, systems and methods for visualizing needle tracts that require cauterization are desirable.

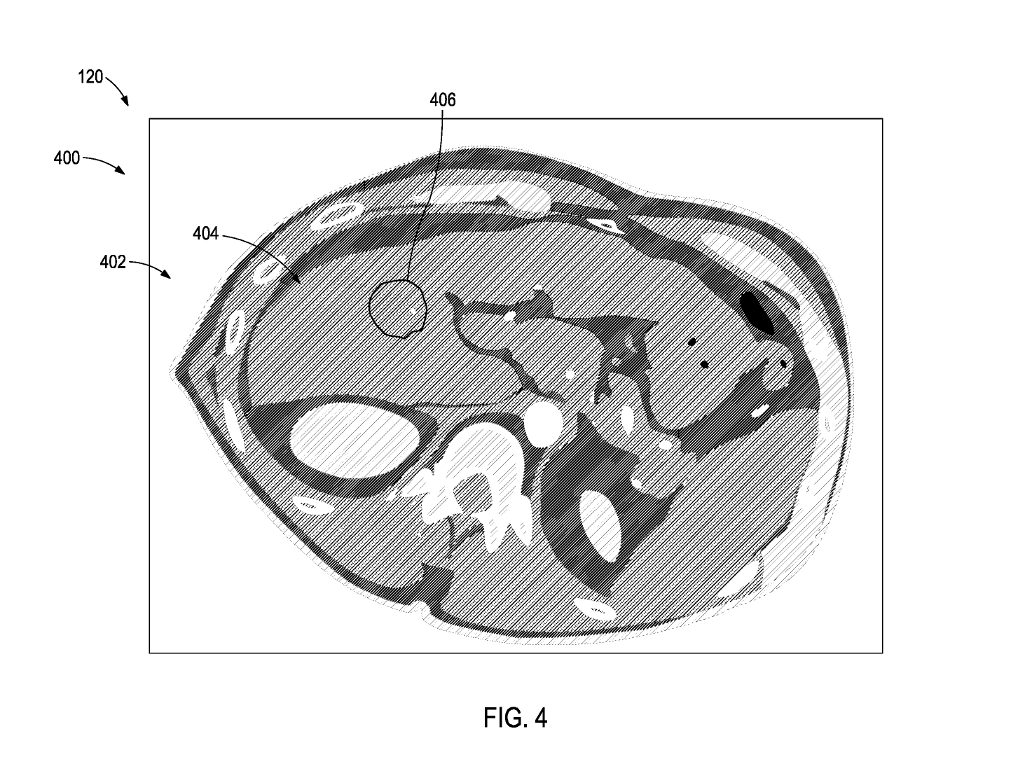

FIG. 4 is a first example image 400 of a patient 402 shown on the display of FIG. 1, according to at least one aspect of the present disclosure. As illustrated, the first image 400 is a CT image of a cross-section of the patient 402, which may be captured (obtained) by the imaging device 119 (FIG. 1) and displayed on the display 120 by the controller 114 (FIG. 1). While a CT image is shown and described, the first image 400 may alternatively be an MRI image, an ultrasound image, a nuclear medicine image, or a fluoroscopy image, or any other suitable image described elsewhere herein. The first image 400 may also be a two-dimensional or three-dimensional image.

The first image 400 shows a body cavity 404 of the patient 402. With reference to FIGS. 1 and 4, a user may determine that there is a region of the body cavity 404 that is to be ablated with an ablation probe 104. Accordingly, the user may provide an input to the controller 114 indicative of a desired region to ablate. For instance, the user may digitally draw on the image 400 a desired region to ablate, such as with a mouse or stylet communicably coupled to the controller 114. Based on receiving the input indicative of the desired region to ablate, the controller 114 may overlay on the displayed image 400 an ablation target 406 representative of the desired region to ablate.

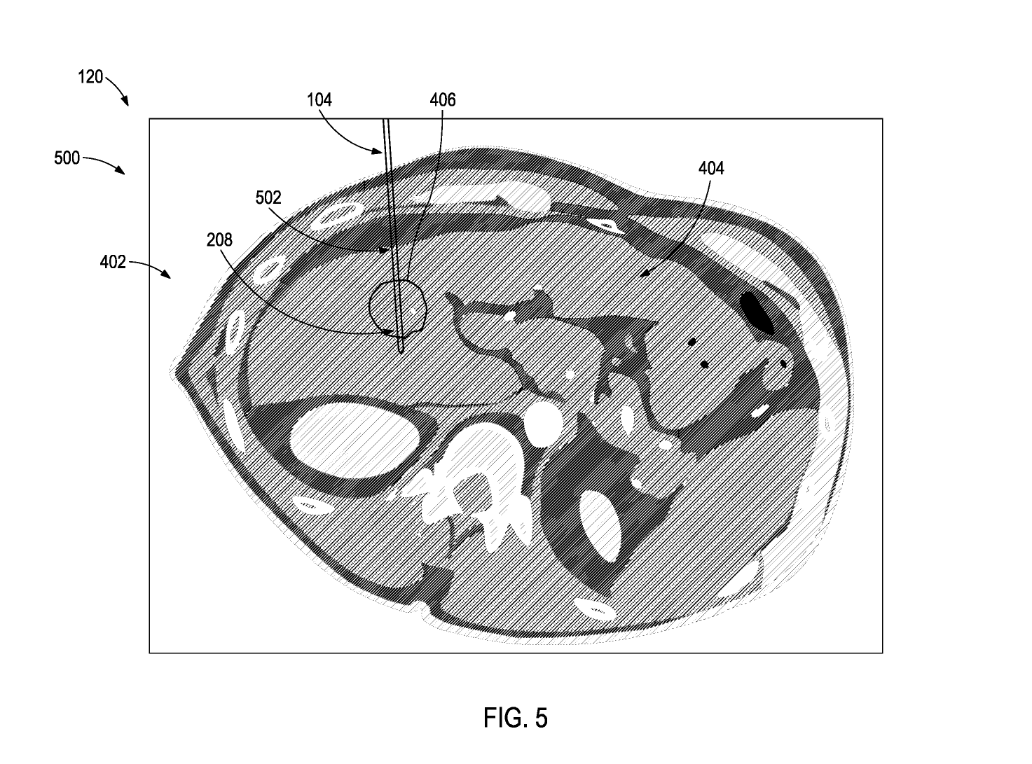

Based on determining a desired region to ablate (e.g. the ablation target 406), the user may insert (place) an ablation probe 104 within the body cavity 404 of the patient 402 and advance the ablation probe 104 toward the ablation target 406. For instance, the user may percutaneously insert the antenna 208 through the skin of the patient 402 to a first position, thereby generating a needle tract within the patient. Once the antenna 208 is placed within the patient at the first position, the controller 114 may then obtain a second image 500 of the patient 402 via the imaging device 119, as shown in FIG. 5. As illustrated, the second image 500 shows an image representative of the ablation probe 104 positioned at a first position along a needle tract 502 and extending to or adjacent the ablation target 406. The position of the ablation probe 104 within the patient 402 may be detected by image recognition software, which may be stored in the memory 117, or may be determined by the controller 114 based on the image captured by the imaging device 119, or a combination thereof.

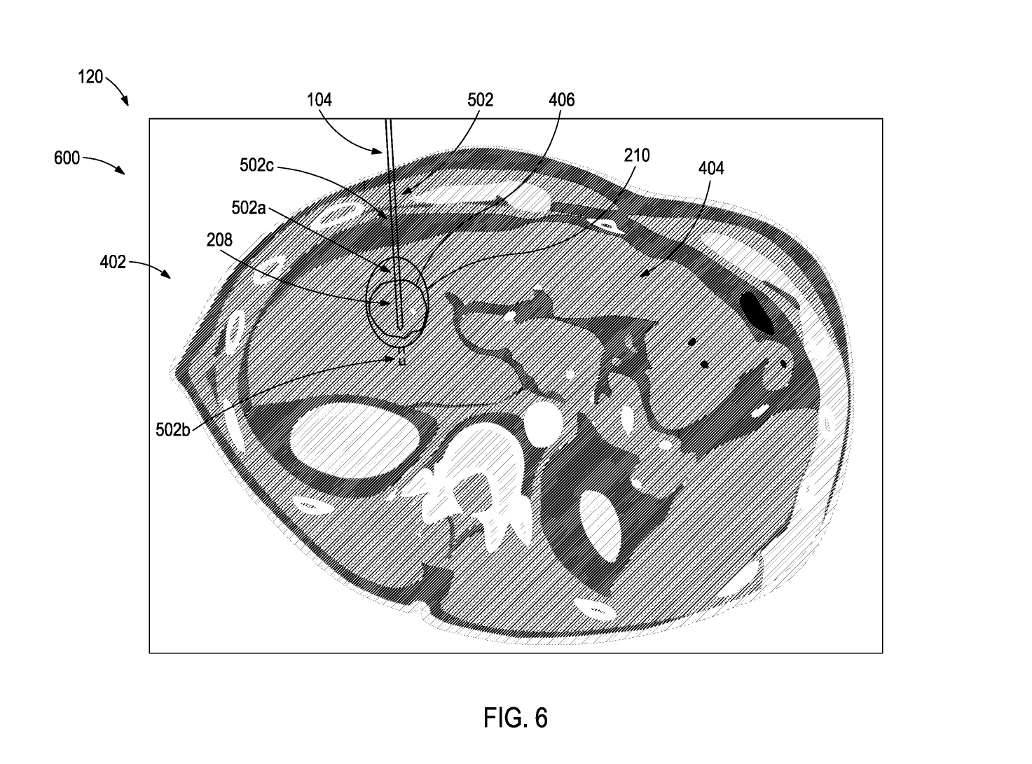

With the antenna 208 at the first position, a user may decide to move the ablation probe 104 from the first position along the needle tract 502 to a second position along the needle tract 502. For instance, as shown in FIG. 5, a user viewing the second image 500 may recognize that the distal end of the antenna 208 (e.g. the stylet 218 (FIG. 2) of the antenna 208) has been inserted (advanced) beyond the ablation target 406. Accordingly, the user may desire to retract (move) the antenna 208 along the needle tract 502 to a different position. Once the user moves the antenna 208 to the second position along the needle tract 502, the controller 114 may obtain a third image 600 of the patient 402 via the imaging device 119, as shown in FIG. 6. As illustrated, the third image 600 shows the ablation probe 104 repositioned along the needle tract 502 at the second position.

With the antenna 208 in the second position, a user may decide to proceed with operating the ablation probe 104 in an ablation mode, in which the antenna 208 generates an ablation zone 210, thereby ablating the ablation target 406. The user may provide to the controller 114 one or more operating parameters for the ablation probe 104, which may include a type of the ablation probe, a desired activation time of the ablation probe, or a power level of the ablation probe, or combinations thereof. As discussed herein, the memory 117 may store therein a look-up table that includes various combinations of types of ablation probes, activation times, and power levels that are expected to yield various sized ablation zones 210 with varying dimensions (e.g. d₁, d₂, d₃; FIG. 2). The expected, or predicted, dimensions of the ablation zone 210 may be based on ex-vivo data, in-vivo data, or clinical data, or combinations thereof. Once provided with the operating parameters of the ablation probe 104, the controller 114 may retrieve from the memory 117 the expected size of the ablation zone 210 based on the operating parameters and may overlay on the display 120 the expected ablation zone 210 for the user to visualize.

As illustrated in FIG. 6, the expected ablation zone 210 is expected to encompass a first portion 502a of the needle tract 502 (i.e. a portion of the needle tract 502 within the bounds of the expected ablation zone 210). The expected ablation zone 210, however, does not encompass a second (distal) portion 502b of the needle tract 502 (i.e. a portion of the needle tract 502 outside of the bounds of the expected ablation zone 210 and encompassing portions of the needle tract 502 occupied by the antenna 208 in the first position; see FIG. 5), and a third (proximal) portion 502c of the needle tract 502 (i.e. a portion of the needle tract 502 outside the bounds of the expected ablation zone 210 in which the shaft 204 (FIG. 2) of the ablation probe 104 still resides).

Based on providing the operating parameters to the controller 114, the user may initiate an ablation treatment, thereby generating the ablation zone 210 within the patient. For instance, the user may provide an input to the controller 114 via a touchscreen of the display 120 to operate the antenna 208 in the ablation mode and generate the ablation zone 210.

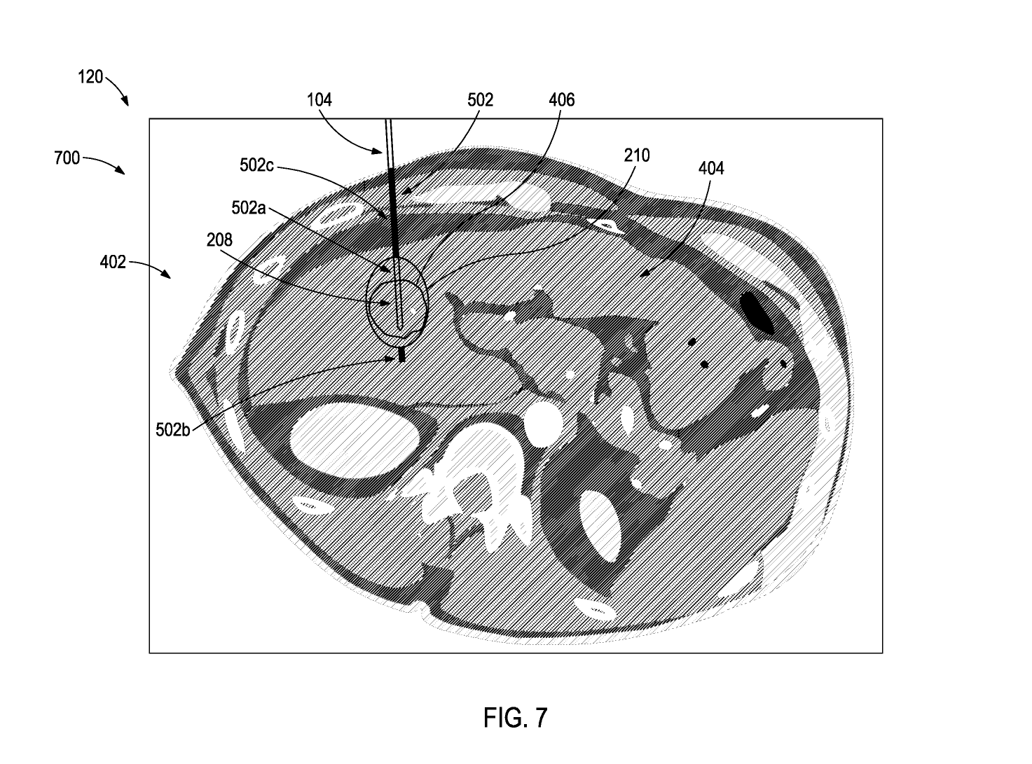

After operating the antenna 208 in the ablation mode, the controller 114 may be configured and otherwise programmed to display regions of the needle tract 502 that may have failed to receive energy from the antenna 208 and, therefore, may be susceptible to tumor seeding. For instance, with reference now to FIG. 7, at the conclusion of operating the antenna 208 in the ablation mode, the controller 114 may display on the image 600 the first and third portions 502a, 502c of the needle tract 502 in a first manner that enables a user to visualize portions of the needle tract 502 that may have failed to receive energy from the antenna 208 (i.e. portions of the needle tract 502 outside the bounds of the expected ablation zone 210). The controller 114 may determine portions of the needle tract 502 to display based on current and previous positions of the antenna 208 along the needle tract 502 and the size of the expected ablation zone 210.

For instance, the controller 114 may compare the current image (e.g. image 600) to the previous image (e.g. image 500) to determine that the antenna 208 was previously positioned within the second portion 502b of the needle tract 502, which was not encompassed (covered) by the expected ablation zone 210. Accordingly, the controller 114 may display the second portion 502b in a first manner that enables a user to visualize that the second portion 502b may have failed to receive energy from the antenna 208. Emphasizing or displaying the second portion 502b in the first manner may include coloring or highlighting the second portion 502b a first color (e.g. highlighting the second portion 502b in red). In the alternative, or in combination therewith, emphasizing or displaying the second portion 502b in the first manner may include displaying the second portion 502b as a first pattern, flashing the second portion 502b, pulsing the second portion 502b, or generating a message on the display 102 that includes a pointer that points to the second portion 502b, or combinations thereof. Similarly, by comparing the current image (e.g. image 600) and the previous image (e.g. image 500), the controller 114 may determine that the expected ablation zone 210 did not encompass the third portion 502c of the needle tract 502 where the shaft 204 (FIG. 2) of the ablation probe 104 currently resides. Accordingly, the controller 114 may be programmed to further display the third portion 502c in the first manner to enable a user to visualize that the third portion 502c may have failed to receive energy from the antenna 208.

After operating the antenna 208 in the ablation mode, the user may transition the ablation probe 104 from the ablation mode to a cauterization mode in which the antenna 208 is slowly withdrawn from the patient 402 while continuously emitting cauterization energy (e.g. microwave energy or RF energy). The user may transition to the cauterization mode by providing an input to the controller 114, such as via a touchscreen of the display 120. With the ablation probe 104 in the cauterization mode, the user may retract the antenna from the patient 402 along the needle tract 502, thereby providing energy to the third portion 502c of the needle tract 502 that prevents tumor seeding.

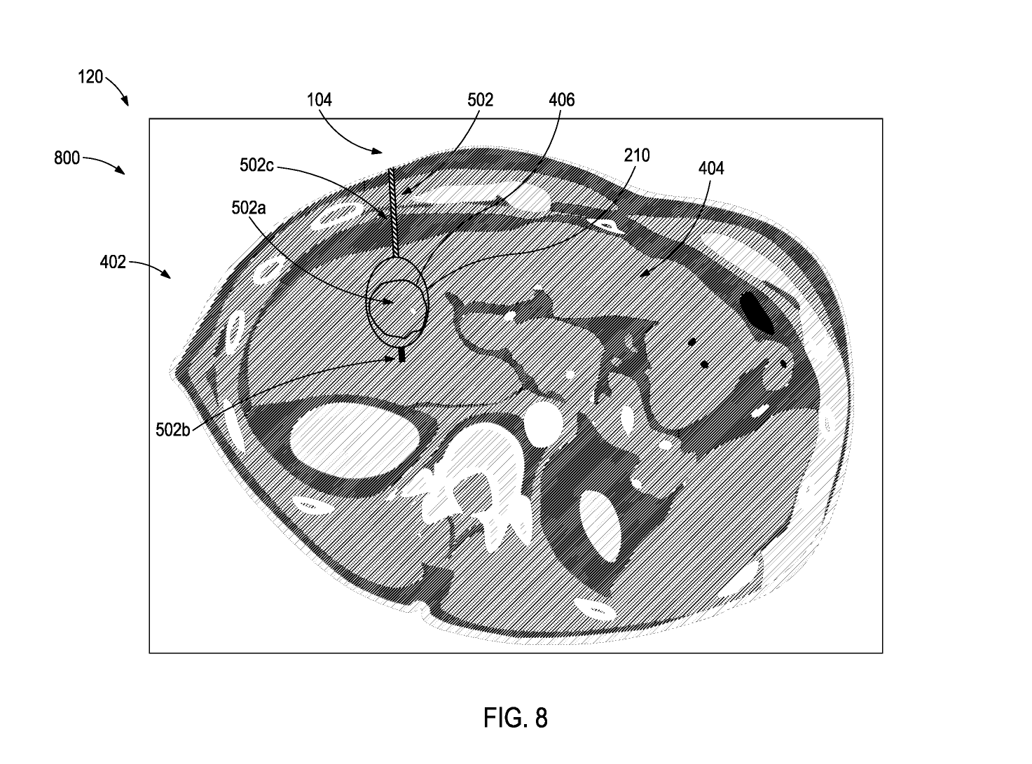

After withdrawing the antenna 208 from the patient 402, the controller 114 may be configured and otherwise programmed to obtain a fourth image 800 of the patient 402 via the imaging device 119, as shown in FIG. 8. As illustrated, the fourth image 800 shows the expected ablation zone 210 having encompassed the first portion 502a of the needle tract 502. The fourth image 800 further shows the second portion 502b of the needle tract 502, which may (still) be displayed by the controller 114 as having failed to receive energy from the antenna 208 and, thus, may be susceptible to tumor seeding. Similar to above, the controller 114 may be configured to display the second portion 502b based on a comparison of the current image (e.g. image 800) and previous images (e.g. images 500, 600) and a location/size of the expected ablation zone 210.

Based on the controller 114 detecting that the antenna 208 has been retracted (moved) through the third portion 502c in the cauterization mode, thereby providing energy to the third portion 502c of the needle tract 502, the controller 114 may display the third portion 502c in a second manner different than the first manner, which enables a user to visualize that the third portion 502c is expected to have received energy from the antenna 208 and thus eliminated the possibility of tumor seeding along that portion. Emphasizing or displaying the third portion 502c in the second manner may include coloring or highlighting the third portion 502c a second color that is different than the first color (e.g. highlighting the second portion 502b red (first color) and highlighting the third portion 502c yellow (second color)). In the alternative, or in combination therewith, emphasizing or displaying the third portion 502c in the second manner may include displaying the third portion 502c as a second pattern different than the first pattern, flashing the third portion 502c, pulsing the third portion 502c, or generating a message on the display 102 that includes a pointer that points to the third portion 502c, or combinations thereof.

Accordingly, the controller 114 enables a user to visualize portions of a needle tract that may have failed to receive energy from an ablation probe and may, therefore, be susceptible to tract tumor seeding.

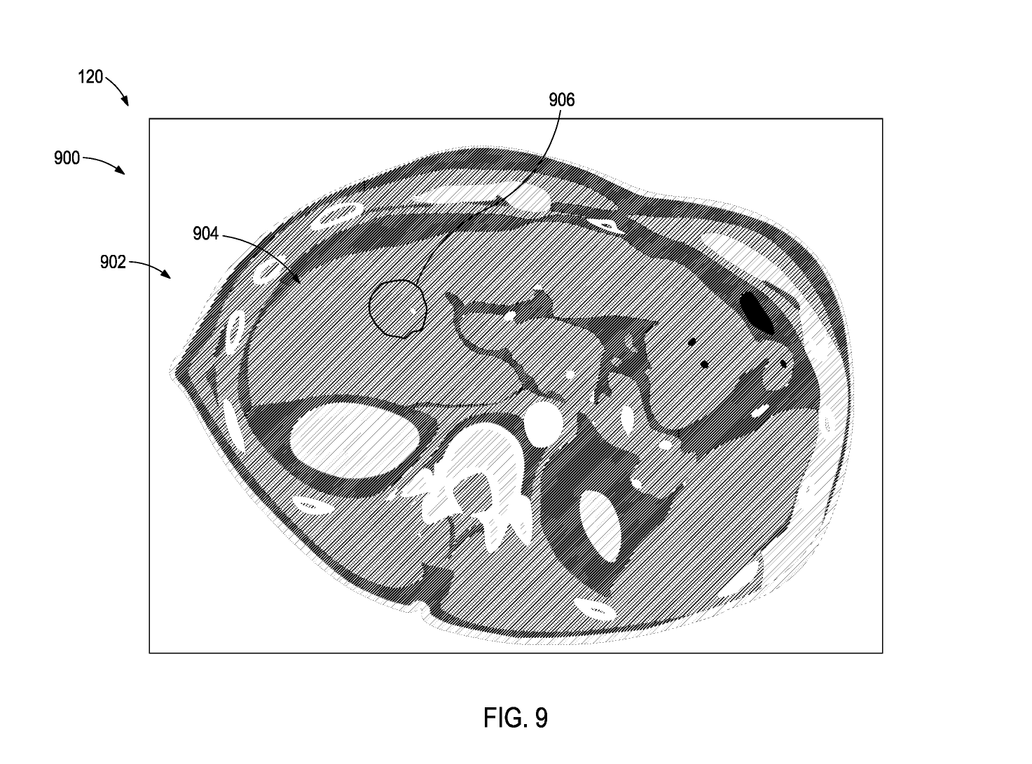

FIG. 9 is a first example image 900 of a patient 902 shown on the display of FIG. 1, according to at least one aspect of the present disclosure. As illustrated, the first image 900 is a CT image of the patient 902, which may be captured (obtained) by the imaging device 119 (FIG. 1) and displayed on the display 120 by the controller 114 (FIG. 1). While a CT image is shown and described, the first image 900 may alternatively be an MRI image, an ultrasound image, a nuclear medicine image, or a fluoroscopy image, or any other suitable image described elsewhere herein. The first image 400 may also be a two-dimensional or three-dimensional image.

The first image 900 shows a body cavity 904 of the patient 902. With reference to FIGS. 1 and 9, a user may determine that there is a region of the body cavity 404 that is to be ablated with an ablation probe 104. Accordingly, the user may provide an input to the controller 114 indicative of a desired region to ablate. For instance, the user may digitally draw on the image 900 a desired region to ablate, such as with a mouse or stylet communicably coupled to the controller 114. Based on receiving the input indicative of the desired region to ablate, the controller 114 may overlay on the displayed image 900 an ablation target 906 representative of the desired region to ablate.

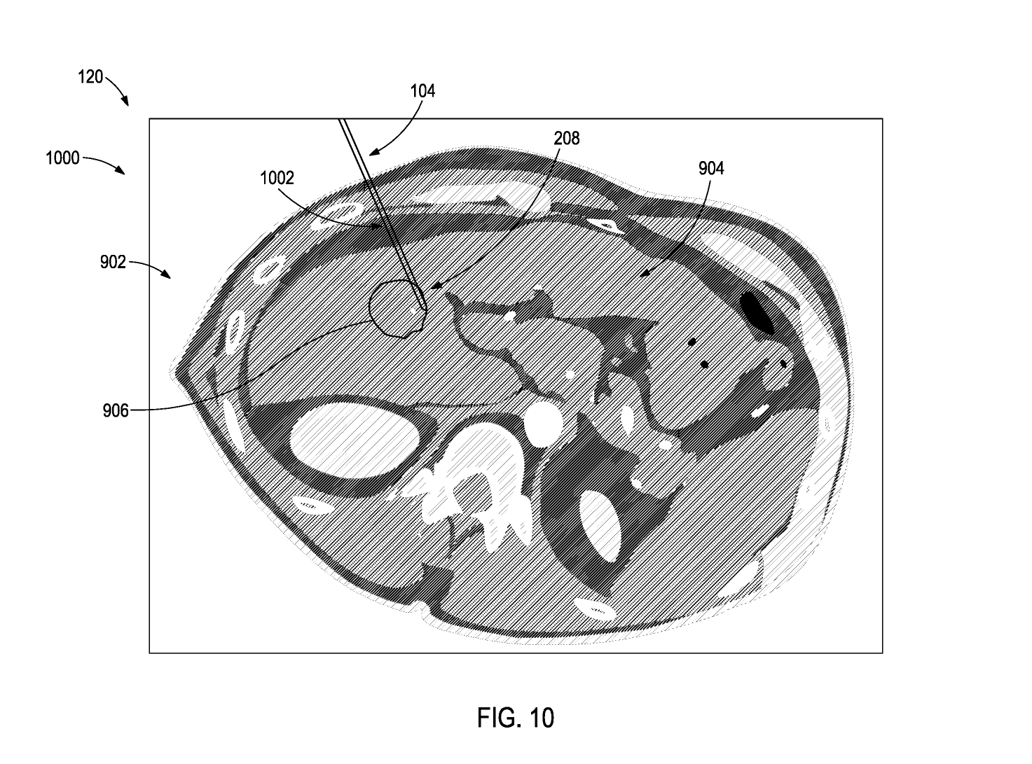

Once the desired region to ablate (e.g. the ablation target 906) is determined, the user may percutaneously insert the antenna 208 of the ablation probe 104 through the skin of the patient 902, thereby generating a first needle tract within the patient. Once the antenna 208 is placed within the patient in the first needle tract, the controller 114 may then obtain a second image 1000 of the patient 902 via the imaging device 119, as shown in FIG. 10. As illustrated, the second image 1000 shows an image representative of the ablation probe 104 positioned along a first needle tract 1002 and adjacent to the ablation target 906. The position of the ablation probe 104 within the patient 902 may be detected by image recognition software stored in the memory 117, or may be determined by the controller 114 based on the image captured by the imaging device 119, or a combination thereof.

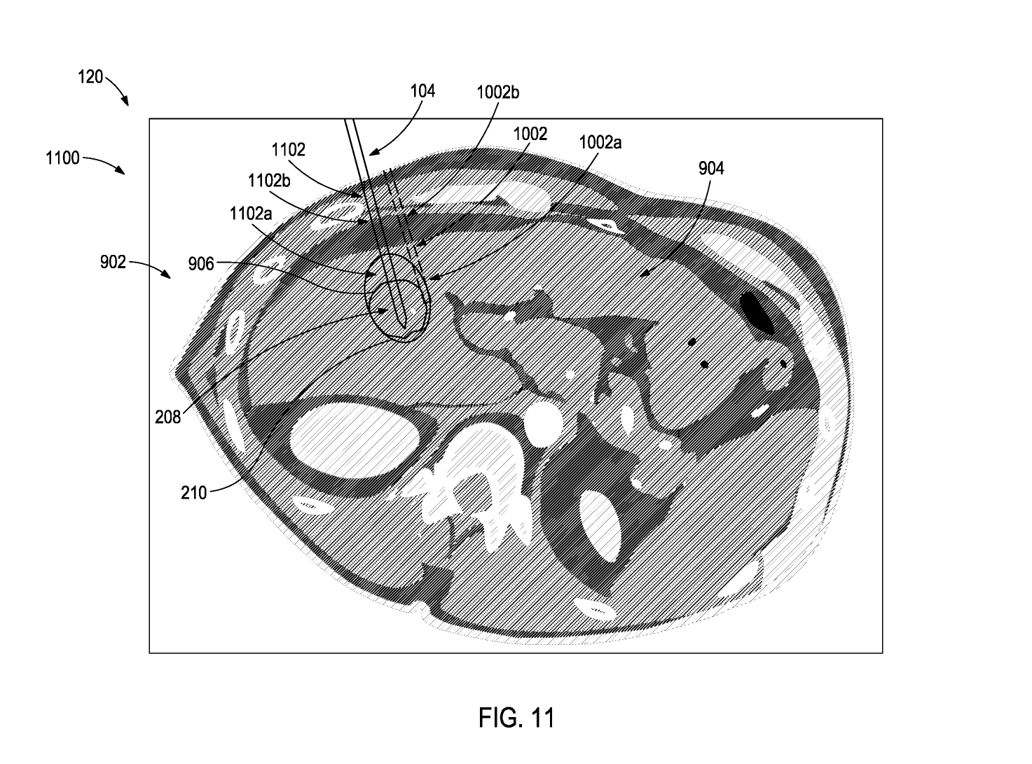

In some scenarios, the user may be unsatisfied with the position of the antenna 208 and may, therefore, decide to withdraw the ablation probe 104 from the first needle tract and re-insert the ablation probe 104 within the body cavity 904 along a second needle tract different than the first needle tract 1002. For instance, the user may be unsatisfied with the position of the antenna 208 relative to the ablation target 906. Accordingly, the user may retract and remove the ablation probe 104 from the needle tract 1002 and re-insert the ablation probe 104 along a second needle tract different than the first needle tract, such as by percutaneously inserting the antenna 208 through a different portion (location) of the skin. Once the antenna 208 is moved to the second needle tract, the controller 114 may obtain a third image 1100 of the patient 902 via the imaging device 119, as shown in FIG. 11. As illustrated, the third image 1100 shows the ablation probe 104 repositioned to a second needle tract 1102.

With the antenna 208 in the second needle tract 1102, a user may decide to proceed with operating the ablation probe 104 in ablation mode, in which the antenna 208 generates an ablation zone 210, thereby ablating the ablation target 906. In particular, the user may provide to the controller 114 one or more operating parameters for the ablation probe 104, which may include a type of the ablation probe, a desired activation time of the ablation probe, or a power level of the ablation probe, or combinations thereof. As discussed herein, the memory 117 may store therein a look-up table that includes various combinations of types of ablation probes, activation times, and power levels that are expected to yield various sized ablation zones 210 with varying dimensions (e.g. d₁, d₂, d₃; FIG. 2). The expected, or predicted, dimensions of the ablation zone 210 may be based on ex-vivo data, in-vivo data, clinical data, or combinations thereof. Once the operating parameters are provided, the controller 114 may retrieve from the memory 117 the expected size of the ablation zone 210 that is expected to be generated based on the provided operating parameters and may overlay on the display 120 the expected ablation zone 210 for the user to visualize.

As illustrated in FIG. 11, the ablation zone 210 is expected to encompass a first portion 1102a of the second needle tract 1102 (i.e. a portion of the second needle tract 1102 within the bounds of the expected ablation zone 210), but is not expected to encompass a second (proximal) portion 1102b of the second needle tract 1102 (i.e. a portion of the second needle tract 1102 outside the bounds of the expected ablation zone 210 in which the shaft 204 (FIG. 2) of the ablation probe 104 still resides). In addition, the expected ablation zone 210 is expected to encompass a first portion 1002a of the first needle tract 1002 (i.e. a portion of the first needle tract 1002 within the bounds of the expected ablation zone 210), but is not expected to encompass a second (proximal) portion 1002b of the first needle tract 1002 (i.e. a portion of the first needle tract 1002 outside the bounds of the expected ablation zone 210 where the ablation probe 104 previously resided).

Based on providing the operating parameters to the controller 114, the user may initiate the ablation treatment, thereby generating the ablation zone 210 within the patient. For instance, the user may provide an input to the controller 114 via a touchscreen of the display 120 to operate the antenna 208 in the ablation mode and generate the ablation zone 210.

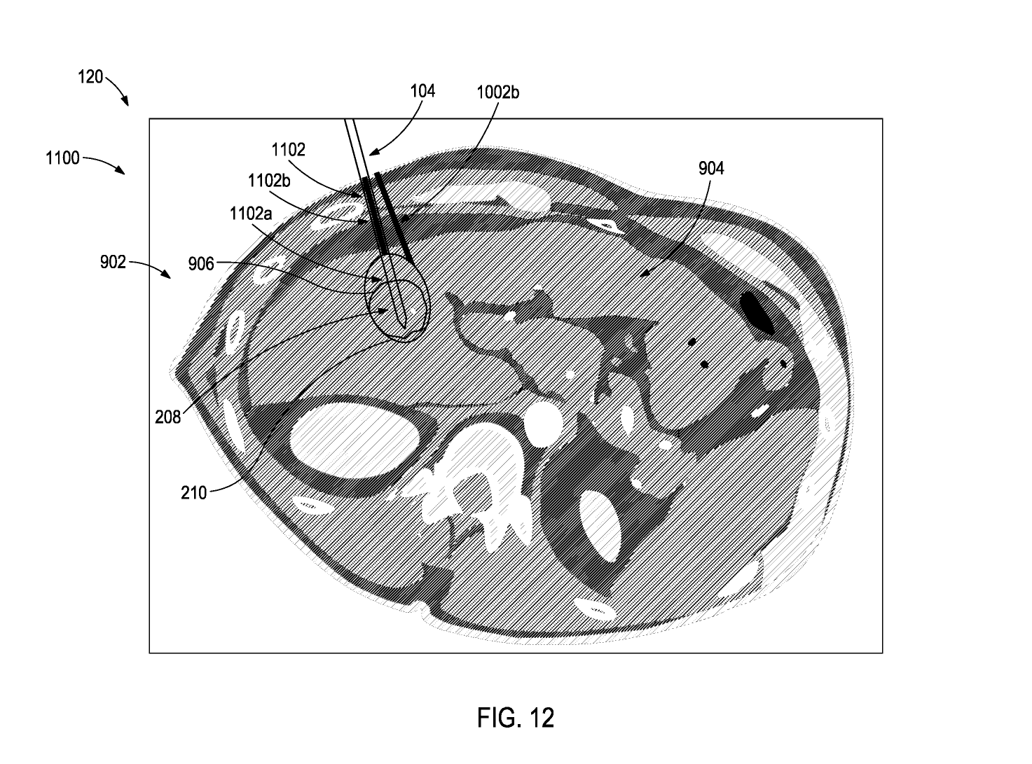

After operating the antenna 208 in the ablation mode, the controller 114 may be configured or otherwise programmed to display regions of the needle tracts 1002, 1102 that failed to receive energy from the antenna 208 and may, therefore, be susceptible to tumor seeding. For instance, with reference now to FIG. 12, at the conclusion of operating the antenna 208 in the ablation mode, the controller 114 may display on the image 1100 the second portions 1002b, 1102b of the first and second needle tracts 1002, 1102 in a first manner that enables the user to visualize portions of the first and second needle tracts 1002, 1102 that failed to receive energy from the antenna 208. The displayed portions of the first and second needle tracts 1002, 1102 are based on current and previous positions of the antenna 208 along the needle tracts 1002, 1102 and the size of the expected ablation zone 210.

For instance, the controller 114 may compare the current image (e.g. image 1100) to the previous image (e.g. image 1000) to determine that the antenna 208 was previously positioned within the second portion 1002b of the first needle tract 1002, which was not encompassed by the expected ablation zone 210. Accordingly, the controller 114 may display the second portion 1002b in the first manner, thus enabling the user to visualize that the second portion 1002b failed to receive energy from the antenna 208. Emphasizing or displaying the second portion 1002b in the first manner may include coloring or highlighting the second portion 1002b a first color (e.g. highlighting the second portion 1002b in red). Similarly, the controller 114 may compare the current image (e.g. image 1000) and previous image (e.g. image 1000) to determine that the expected ablation zone 210 is not expected to encompass the second portion 1102b of the second needle tract 1102 where the shaft 204 (FIG. 2) of ablation probe 104 currently resides. Accordingly, the controller 114 may display the second portion 1102b in the second manner to notify the user that the second portion 1102b may not have received energy from the antenna 208.

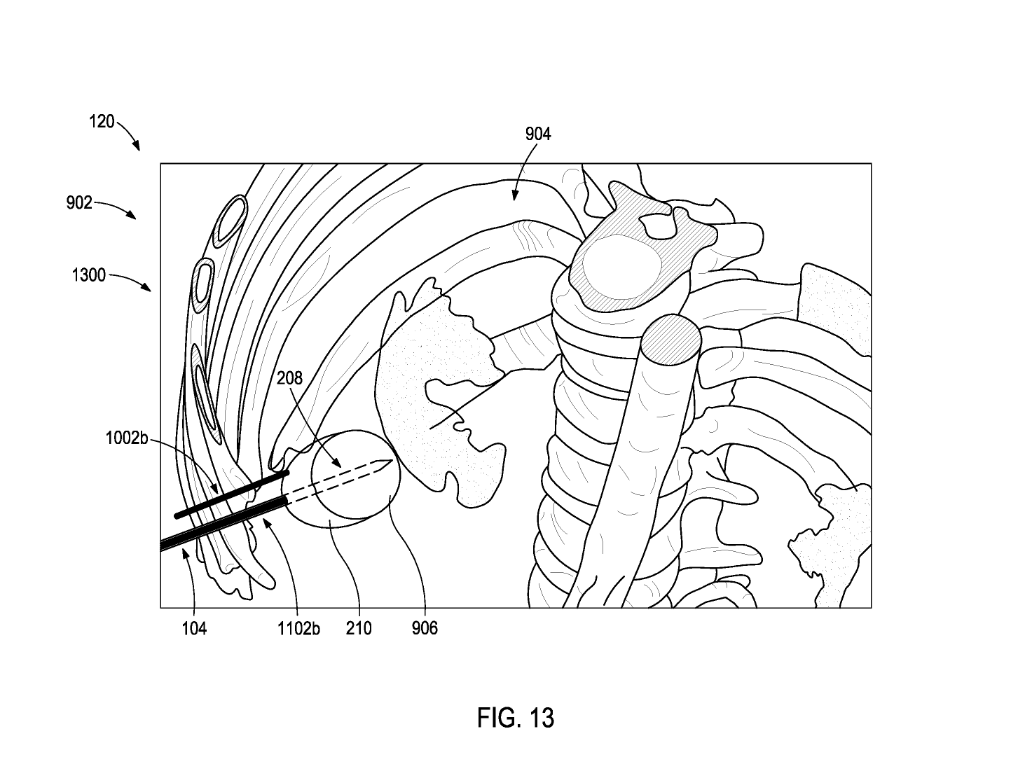

In some embodiments, the user may provide an input to the controller 114, such as via a touchscreen of the display 120, to transition the third image 1100 (i.e. a two-dimensional image) to a fourth, three-dimensional image 1300, as shown in FIG. 13. In the three-dimensional image 1300, the user is able to visualize portions of the first and second needle tracts 1002, 1102 that are expected to have failed to receive energy from the antenna 208. The fourth image 1300 may be captured by the imaging device 119 and provided to the controller 114.

After operating the antenna 208 in the ablation mode, the user may transition the ablation probe 104 from the ablation mode to a cauterization mode in which the antenna 208 may emit cauterization energy (e.g. microwave energy or RF energy) as the ablation probe 104 is withdrawn. The user may transition the ablation probe 104 from the ablation mode to the cauterization mode by providing an input to the controller 114, such as via a touchscreen of the display 120. With the ablation probe 104 in the cauterization mode, the user may retract the antenna 208 from the patient 902 along the second needle tract 1102, thereby providing energy to the second portion 1102b of the second needle tract 1102.

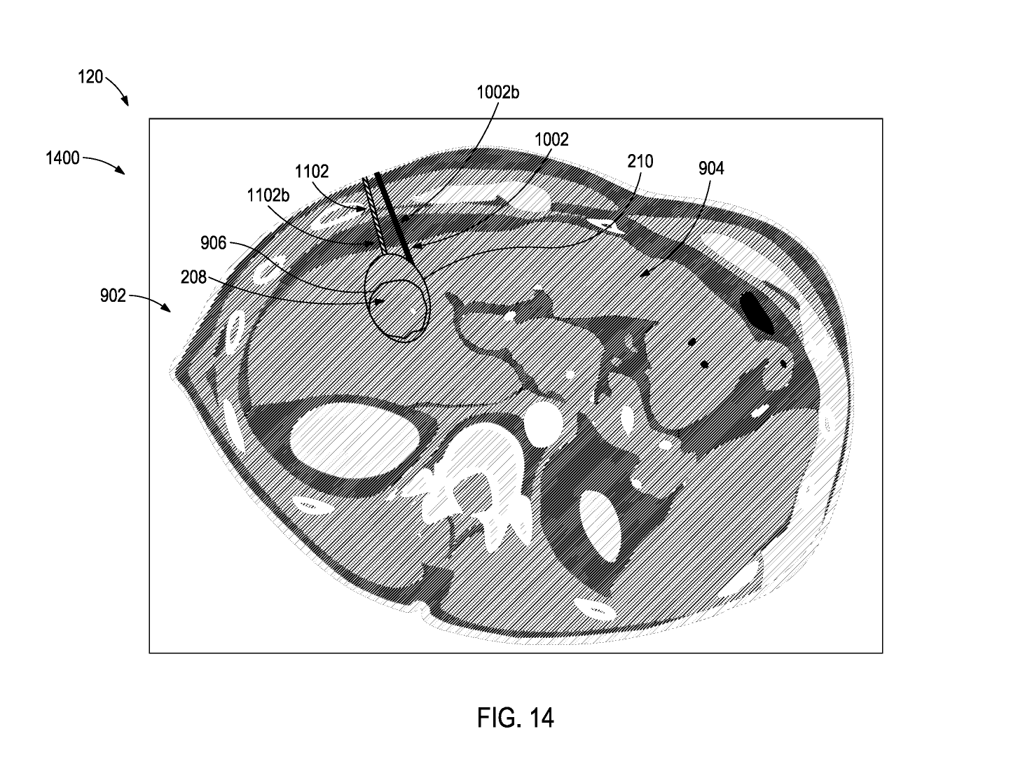

After withdrawing the antenna 208 from the patient 902, the controller 114 may obtain a fifth image 1400 of the patient 902 via the imaging device 119, as shown in FIG. 14. As illustrated, the fifth image 1400 shows the expected ablation zone 210, which is expected to have encompassed the first portion 1002a (FIG. 11) of the first needle tract 1002 and the first portion 1102a (FIG. 11) of the second needle tract 1102. The fifth image 1400 further shows the second portion 1002b of the first needle tract 1002, which may (still) be displayed by the controller 114 as expecting to have failed to receive energy from the antenna 208 and, thus, may be susceptible to tumor seeding. The controller 114 may be configured to display the second portion 1002b based on a comparison of the current image (e.g. image 1400) and previous images (e.g. images 1000, 1100, 1300) and a location/size of the expected ablation zone 210.

Once the antenna 208 has been retracted (moved) through the second portion 1102b in the cauterization mode, thereby providing energy to the second portion 1102b of the second needle tract 1102, the controller 114 may be configured to display the second portion 1102b in a second manner different than the first manner to notify the user that the second portion 1102b received energy from the antenna 208. Emphasizing or displaying the second portion 1102b in a second manner may include coloring or highlighting the second portion 1102b a second color that is different than the first color (e.g. highlighting the second portion 1002b red (first color) and highlighting the second portion 1102b yellow (second color)).

Accordingly, the foregoing systems and methods enable a user to visualize one or more needle tracts that are expected to have failed to receive energy from an ablation probe and may, therefore, be susceptible to tract seeding. Accordingly, the systems and methods provide a user with a visual indication of portions of needle tracts that should be revisited to provide energy thereto.

### Embodiments disclosed herein include:

A. A system comprising an ablation probe including an antenna and a controller in operable communication with the ablation probe and a display. The controller is operable to display, on the display, an image of the ablation probe in a needle tract in the patient, control the antenna to emit energy in the needle tract, and display, on the image, portions of the needle tract expected to have failed to receive energy from the antenna.

B. A method comprising detecting an ablation probe being positioned in a first needle tract within a patient, detecting the ablation probe being repositioned in a second needle tract within the patient, displaying, on a display, an image of the ablation probe in the second needle tract, controlling an antenna of the ablation probe to emit energy in the second needle tract, and displaying, on the image, portions of the first and second needle tracts expected to have failed to receive energy from the antenna.

C. A non-transitory computer readable medium storing instructions that, when executed by a processor, causes the processor to display, on a display, an image of an ablation probe in a needle tract in a patient, control an antenna of the ablation probe to emit energy in the needle tract, and display, on the image, portions of the needle tract expected to have failed to receive energy from the antenna.

Each of embodiments A, B, and C may have one or more of the following additional elements in any combination: Element 1: wherein to display the portions, the controller is operable to highlight the portions on the image. Element 2: wherein the ablation probe is operable in an ablation mode in which the antenna generates an ablation zone and a cauterization mode in which the antenna emits cauterization energy, wherein to display the portions, the controller is operable to display first portions of the needle tract not expected to have been encompassed by the ablation zone and thereby failed to receive cauterization energy from the antenna. Element 3: wherein the controller is further operable to display, on the image, second portions of the needle tract not expected to have been encompassed by the ablation zone, but expected to have received cauterization energy from the antenna. Element 4: wherein the controller is operable to display the first portions of the needle tract in a first manner and display the second portions of the needle tract in a second manner different than the first manner. Element 5: wherein to display the first portions in the first manner, the controller is operable to display the first portions in a first color and to display the second portions in the second manner, the controller is operable to display the second portions in a second color different than the first color. Element 6: wherein the controller is further operable to detect a previous needle tract in the patient into which the antenna was previously positioned and display, on the image, portions of the previous needle tract expected to have failed to receive energy from the antenna. Element 7: wherein displaying portions of the first and second needle tracts expected to have failed to receive energy from the antenna comprises highlighting portions of the image. Element 8: wherein the ablation probe is operable in an ablation mode in which the antenna generates an ablation zone and a cauterization mode in which the antenna emits cauterization energy, wherein display portions of the first and second needle tracts expected to have failed to receive energy from the antenna comprises displaying first portions of the first and second needle tracts not expected to have been encompassed by the ablation zone and thereby failed to receive cauterization energy from the antenna. Element 9: further comprising displaying, on the image, second portions of the first and second needle tracts not expected to have been encompassed by the ablation zone, but expected to have received cauterization energy from the antenna. Element 10: wherein displaying the first portions of the first and second needle tracts comprises displaying the first portions of the first and second needle tracts in a first manner and displaying the second portions of the first and second needle tracts comprises displaying the second portions of the first and second needle tracts in a second manner different than the first manner. Element 11: wherein displaying the first portions of the first and second needle tracts in the first manner comprises displaying the first portions in a first color and displaying the second portions of the first and second needle tracts in the second manner comprises displaying the second portions in a second color different than the first color. Element 12: wherein to display the portions, the processor is operable to highlight the portions on the image. Element 13: wherein the ablation probe is operable in an ablation mode in which the antenna generates an ablation zone and a cauterization mode in which the antenna emits cauterization energy, wherein to display the portions, the processor is operable to display first portions of the needle tract not expected to have been encompassed by the ablation zone and thereby failed to receive cauterization energy from the antenna. Element 14: further storing instructions that, when executed by the processor, causes the processor to display, on the image, second portions of the needle tract not expected to have been encompassed by the ablation zone, but expected to have received cauterization energy from the antenna. Element 15: further storing instructions that, when executed by the processor, causes the processor to display the first portions of the needle tract in a first manner and display the second portions of the needle tract in a second manner different than the first manner. Element 16: wherein to display the first portions in the first manner, the processor is operable to display the first portions in a first color and to display the second portions in the second manner, the processor is operable to display the second portions in a second color different than the first color. Element 17: further storing instructions that, when executed by the processor, causes the processor to detect a previous needle tract in the patient into which the antenna was previously positioned and display, on the image, portions of the previous needle tract expected to have failed to receive energy from the antenna.

By way of non-limiting example, exemplary combinations applicable to A, B, and C include: Element 1 with Element 2; Element 1 with two or more of Elements 2-6; Element 2 with Element 3; Element 2 with Elements 3 and 4; Element 2 with Elements 3-5; Element 6 with one or more of Elements 1-5; Element 7 with Element 8; Element 7 with two or more of Elements 8-11; Element 8 with Element 9; Element 8 with Elements 9 and 10; Element 8 with Elements 9-11; Element 12 with Element 13; Element 12 with two or more of Elements 13-17; Element 13 with Element 14; Element 13 with Elements 14 and 15; Element 13 with Elements 14-16; Element 17 with one or more of Elements 12-16.

Therefore, the disclosed systems and methods are well adapted to attain the ends and advantages mentioned as well as those that are inherent therein. The particular embodiments disclosed above are illustrative only, as the teachings of the present disclosure may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope of the present disclosure. The systems and methods illustratively disclosed herein may suitably be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces. If there is any conflict in the usages of a word or term in this specification and one or more patent or other documents that may be incorporated herein by reference, the definitions that are consistent with this specification should be adopted.

As used herein, the phrase “at least one of” preceding a series of items, with the terms “and” or “or” to separate any of the items, modifies the list as a whole, rather than each member of the list (i.e., each item). The phrase “at least one of” allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The use of directional terms such as above, below, upper, lower, upward, downward, left, right, and the like are used in relation to the illustrative embodiments as they are depicted in the figures, the upward direction being toward the top of the corresponding figure and the downward direction being toward the bottom of the corresponding figure.

What is claimed is:

1. A system, comprising:
an ablation probe including an antenna; and
a controller in operable communication with the ablation probe and a display, wherein the controller is operable to:
display, on the display, an image of the ablation probe in a needle tract in the patient;
control the antenna to emit energy in the needle tract; and
display, on the image, portions of the needle tract expected to have failed to receive energy from the antenna.

2. The system of claim 1, wherein to display the portions, the controller is operable to highlight the portions on the image.

3. The system of claim 1, wherein the ablation probe is operable in:
an ablation mode in which the antenna generates an ablation zone; and
a cauterization mode in which the antenna emits cauterization energy,
wherein to display the portions, the controller is operable to display first portions of the needle tract not expected to have been encompassed by the ablation zone and thereby failed to receive cauterization energy from the antenna.

4. The system of claim 3, wherein the controller is further operable to display, on the image, second portions of the needle tract not expected to have been encompassed by the ablation zone, but expected to have received cauterization energy from the antenna.

5. The system of claim 4, wherein the controller is operable to:
display the first portions of the needle tract in a first manner; and
display the second portions of the needle tract in a second manner different than the first manner.

6. The system of claim 5, wherein:
to display the first portions in the first manner, the controller is operable to display the first portions in a first color; and
to display the second portions in the second manner, the controller is operable to display the second portions in a second color different than the first color.

7. The system of claim 1, wherein the controller is further operable to:
detect a previous needle tract in the patient into which the antenna was previously positioned; and
display, on the image, portions of the previous needle tract expected to have failed to receive energy from the antenna.

8. A method, comprising:
detecting an ablation probe being positioned in a first needle tract within a patient;
detecting the ablation probe being repositioned in a second needle tract within the patient;
displaying, on a display, an image of the ablation probe in the second needle tract;
controlling an antenna of the ablation probe to emit energy in the second needle tract; and
displaying, on the image, portions of the first and second needle tracts expected to have failed to receive energy from the antenna.

9. The method of claim 8, wherein displaying portions of the first and second needle tracts expected to have failed to receive energy from the antenna comprises highlighting portions of the image.

10. The method of claim 8, wherein the ablation probe is operable in:
an ablation mode in which the antenna generates an ablation zone; and
a cauterization mode in which the antenna emits cauterization energy,
wherein display portions of the first and second needle tracts expected to have failed to receive energy from the antenna comprises displaying first portions of the first and second needle tracts not expected to have been encompassed by the ablation zone and thereby failed to receive cauterization energy from the antenna.

11. The method of claim 10, further comprising displaying, on the image, second portions of the first and second needle tracts not expected to have been encompassed by the ablation zone, but expected to have received cauterization energy from the antenna.

12. The method of claim 11, wherein:
displaying the first portions of the first and second needle tracts comprises displaying the first portions of the first and second needle tracts in a first manner; and
displaying the second portions of the first and second needle tracts comprises displaying the second portions of the first and second needle tracts in a second manner different than the first manner.

13. The method of claim 12, wherein:
displaying the first portions of the first and second needle tracts in the first manner comprises displaying the first portions in a first color; and
displaying the second portions of the first and second needle tracts in the second manner comprises displaying the second portions in a second color different than the first color.

14. A non-transitory computer readable medium storing instructions that, when executed by a processor, causes the processor to:
display, on a display, an image of an ablation probe in a needle tract in a patient;
control an antenna of the ablation probe to emit energy in the needle tract; and
display, on the image, portions of the needle tract expected to have failed to receive energy from the antenna.

15. The non-transitory computer readable medium of claim 14, wherein to display the portions, the processor is operable to highlight the portions on the image.

16. The non-transitory computer readable medium of claim 14, wherein the ablation probe is operable in:
an ablation mode in which the antenna generates an ablation zone; and
a cauterization mode in which the antenna emits cauterization energy,
wherein to display the portions, the processor is operable to display first portions of the needle tract not expected to have been encompassed by the ablation zone and thereby failed to receive cauterization energy from the antenna.

17. The non-transitory computer readable medium of claim 16, further storing instructions that, when executed by the processor, causes the processor to display, on the image, second portions of the needle tract not expected to have been encompassed by the ablation zone, but expected to have received cauterization energy from the antenna.

18. The non-transitory computer readable medium of claim 17, further storing instructions that, when executed by the processor, causes the processor to:
display the first portions of the needle tract in a first manner; and
display the second portions of the needle tract in a second manner different than the first manner.

19. The non-transitory computer readable medium of claim 18, wherein:
to display the first portions in the first manner, the processor is operable to display the first portions in a first color; and
to display the second portions in the second manner, the processor is operable to display the second portions in a second color different than the first color.

20. The non-transitory computer readable medium of claim 14, further storing instructions that, when executed by the processor, causes the processor to:
detect a previous needle tract in the patient into which the antenna was previously positioned; and
display, on the image, portions of the previous needle tract expected to have failed to receive energy from the antenna.
